(19) United States
(12) Patent Application Publication
(10) Pub. No.: US 2005/0163670 A1
(43) Pub. Date: Jul. 28, 2005

(54) Heat activated air freshener system utilizing auto cigarette lighter

(76) Inventors: Stephnie Alleyne, Brooklyn, NY (US); Judy Alleyne, Brooklyn, NY (US)

Correspondence Address: Philip M. Weiss;Weiss & Weiss, 310 Old Country Road, Garden City, NY, 11530, US

(21) Appl. No.: 10/753,734

(22) Filed: Jan. 8, 2004

(51) Int. Cl. A61L 9/03

(52) U.S. Cl. 422/125; 422/124

(57) ABSTRACT

A heat activated air freshener system utilizing auto cigarette lighter.

### FIELD OF THE INVENTION

The present invention relates to a heat activated air freshener system utilizing auto cigarette lighter.

### BACKGROUND OF THE INVENTION

U.S. Pat. No. 4,377,399 relates to an air freshening device comprising an upright housing including, adjacent the bottom thereof, an air inlet, and adjacent the top thereof, an air discharge, a fan in said housing intermediate the air inlet and the air discharge means for causing air flow through the housing, a filter, structure for removably supporting the filter in the housing intermediate the air inlet and the fan in the path of the totality of the air flow through the housing, a sleeve assembly within the housing for introducing into the air flow downstream of the filter a substance to be dispensed, which sleeve assembly is operable to releasably support the filter and includes rotatable inner and outer sleeves, one of the sleeves defining an interior chamber openings in the sleeves for affording valved access to the interior chamber in response to relative rotation between the sleeves, a coupling for releasably connecting the sleeve assembly and the housing, and a package of the substance to be dispensed located in the interior chamber.

U.S. Pat. No. 4,523,870 relates to an aroma-dispensing cartridge and holder assembly for automobiles which attach to the air vent in the interior of an automobile. When activated, the assembly functions to diffuse an aromatic vapor into the forced air stream emitted through the vent into the interior. The assembly is constituted by a holder provided with an array of parallel slots and a replaceable cartridge which is telescoped therein. The cartridge contains a porous pad impregnated with a liquid scent and includes a like array of slots. The inserted cartridge is axially shiftable relative to the holder from an inactive position in which the holder an cartridge slots are out of registration to effectively seal the pad, to an active position in which the slots lie in registration, as a consequence of which the forced air stream from the vent passes through the porous pad to volatize the liquid scent.

U.S. Pat. No. 4,339,079 relates to a fragrance emitter in a room or automobile. The device includes a housing that encloses a container of a fragrant liquid. Part of the container is made of a transparent material that can be pierced by a needle.

U.S. Pat. No. 6,264,887 relates to a device for allowing perfumed or deodorizing materials to the surrounding air of a vehicle, which includes a clip device adapted to be connected to an air vent fin. The device is adapted to be inserted into the vent.

U.S. Pat. No. 4,808,347 relates to a fan driven air freshener for confined spaces. The device includes a housing which has an elongated cylindrical rear section adapted to plug into a standard cigarette lighter socket. An electric motor within the front section of the housing contains a propeller fastened to its shaft, the motor being powered through electrical contacts protruding through the rear section of the housing and contacting complementary contacts within the socket. A perforated dome shaped cover removably fastenable to the larger diameter front section of the housing containing the propeller has a concave interior space adapted to hold a disposable scent producing an odor absorbing perforated disc.

U.S. Pat. No. 4,968,456 relates to an electrical air freshener for cars. The air freshener is plugged into an automobile cigarette lighter receptacle. The air freshener includes a housing; an electrical motor within the housing connectable to a source of electrical current, a switch for switching on and off the current; air flow inlets; odor emitting means and a fan. A stem protrudes from the back of the housing and is attached for insertion into the cigarette lighter receptacle.

U.S. Pat. No. 6,197,263 relates to an air freshener for cars having a base-power unit and a detachable fragrance dispensing unit. The fragrance dispensing unit has a heating element disposed therein and is adapted to receive standard replaceable gel-scent cartridges. The detachable fragrance dispensing unit can be attached to an automobile vent system to utilize the car's air moving system to disperse the fragrance throughout the car. The power supply in the base power unit allows a user to select between drawing power from a cigarette lighter plug, a battery disposed within the base power unit, or a solar electric cell disposed on the outer surface of the base power unit.

U.S. Pat. No. 5,394,506 relates to a fragrance dispenser for a vehicle, that utilizes the automobile's cigarette lighter as a heating source. The fragrance dispenser includes a housing for plugging it into the cigarette lighter socket and a wedge shaped divergent housing that has an open, top portion that allows for dispensing of the fragrance. The system utilizes a replaceable cartridge of a volatile fragrance emitting material.

### SUMMARY OF THE INVENTION

The present invention relates to a heat activated air freshener system which utilizes a car's cigarette lighter. It is an object of the present invention to have a device that comprises a male insert plug that is inserted into the cigarette lighter. It is an object of the present invention for the air freshener unit to leave an opening which allows for plugins for other devices. It is an object of the present invention for the device to further allow a cell phone to be also plugged in. It is an object of the present invention for the device to allow other devices to utilize the plug while simultaneously activating the freshener.

It is an object of the present invention for the device to take the shape of a fruit or a logo. It is an object of the present invention for the device to comprise an extension cord that allows the freshener to reach the back of an automobile. It is an object of the present invention to have a built in clip fan to assist in distributing the freshener scent.

### BRIEF DESCRIPTION OF THE DRAWINGS

FIG. 1 is a side view of an embodiment of the present invention.

FIG. 2 is a side view of an embodiment of the present invention.

FIG. 3 is a side view of an embodiment of the present invention.

### DETAILED DESCRIPTION OF THE INVENTION

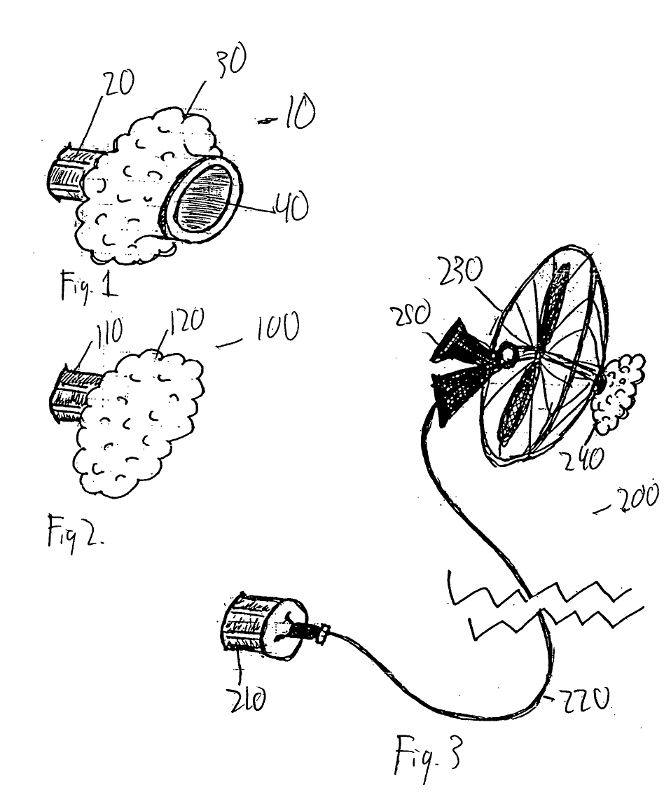

FIG. 1 illustrates an embodiment of the present invention. Device 10 has a male insert plug 20 which is inserted into a cigarette lighter not shown. The device has an air freshener 30 which upon heating of the cigarette lighter, the scent is released into the automobile. The device 10 has an opening 40 which allows other devices to be plugged in.

FIG. 2 illustrates an embodiment of the present invention. Device 100 has a male insert plug 110 which is inserted into a cigarette lighter not shown. The device has an air freshener 120 which upon heating of the cigarette lighter, the scent is released into the automobile.

FIG. 3 illustrates an embodiment of the present invention. Device 200 has a male insert plug 210 which is inserted into a cigarette lighter not shown. Male insert plug 210 is attached to an extension cord 220 which is attached to a fan 230. The fan 230 is attached to the air freshener 240. The fan can have a clip 250 so that the fan can be attached to different areas of the automobile.

1. A heat activated air freshener system comprising:
a male insert plug that is inserted into a cigarette lighter;
an air freshener unit;
an opening which allows for plugins for other devices, and allows for said other devices to utilize said plug while simultaneously activating said freshener.

2. The system of claim 1 wherein said opening allows a cell phone to be also plugged in.

3. The system of claim 1 wherein said air freshener is shape of a fruit or logo.

4. The system of claim 1 wherein said device further comprises:
an extension cord that allows said freshener to reach a back of an automobile; and
a fan to assist in distributing said freshener scent.
